(19) United States
(12) Patent Application Publication
(10) Pub. No.: US 2007/0014839 A1
(43) Pub. Date: Jan. 18, 2007

(54) Decomposer film for transdermal patches

(76) Inventor: Stefan Bracht, Glienicke Nordbahn (DE)

Correspondence Address: STRIKER, STRIKER & STENBY, 103 EAST NECK ROAD, HUNTINGTON, NY, 11743, US

(21) Appl. No.: 11/327,823

(22) Filed: Jan. 6, 2006

Related U.S. Application Data
(60) Provisional application No. 60/700,116, filed on Jul. 18, 2005.

(51) Int. Cl. A61K 9/70; A61K 31/56

(52) U.S. Cl. 424/449; 514/182

(57) ABSTRACT

The decomposer film product has a polymeric decomposer layer, a cover layer for protecting the decomposer film from the surroundings and a releasable support liner, which is removed prior to use. The polymeric decomposer film contains a water-soluble or water-insoluble polymeric adhesive material and a decomposition accelerator, which acts to decompose an effective ingredient, such as a steroid hormone, of a worn or unused transderamal patch, when the effective ingredient releasing layer of the patch adheres to the polymer film, so that the pharmaceutical effective ingredient comes into contact with the decomposition accelerator by diffusion. The decomposition accelerator includes a chemically oxidizing substance, preferably urea peroxide, manganese (III) acetate or iron (III) citrate. The water-insoluble polymeric adhesive material is preferably an acrylates adhesive. The water-soluble polymeric adhesive material is preferably polyvinyl alcohol, polyvinyl pyrrolidone, a cellulose derivative or a polyacrylic acid.

### CROSS-REFERENCE

The present invention described hereinbelow is also described in U.S. Provisional Application Ser. No. 60/700,116, filed Jul. 18, 2005. Priority of invention based on the aforesaid U.S. Provisional Application is hereby claimed under 35 U.S.C. 119 (e).

### BACKGROUND OF THE INVENTION

Transdermal patches differ from most other forms of pharmaceutical administration, since they are not taken internally or applied to the body without later removal, but are again removed at the end of their application time by the user. The removed patch currently is typically thrown out in the house garbage. Generally considerable residual pharmaceutically effective ingredients remain in the removed patches. This is due to the fact that an over-supply or excess of the effective ingredient is required in the patch to maintain a uniform effective ingredient flow through the skin during the application time in order to keep the released activity uniformly high. As a result of the controlled administration rate only from 10 to 40% of the effective ingredient is delivered during the application time interval, or 60 to 90% of the effective ingredient remains after a conventional administration time. These residual amounts of effective ingredient are potentially environmentally damaging when they are present in house garbage, especially when the residual effective ingredient in the house garbage decomposes only slowly and is delivered again to the surroundings. Furthermore patch waste with high effective ingredient content is dangerous for children. Also there is a risk of misuse by those with drug dependencies, in the case of strong analgesics.

A discussion of possible environmental risks occurred in the fields of hormone replacement therapy and contraception using steroid hormones due to the wide use of these treatment methods in large sectors of the population. Here one must distinguish between compounds, which are inactivated comparatively rapidly in the environment, such as estradiol, and e.g. its synthetic derivatives, which decompose slowly, such as e.g. ethinyl estradiol (see lit. 1). There is a greater environmental risk from these latter compounds. The official instructions for contraceptive-acting EVRA patches of Ortho-McNeil in the publication by Janssen Cilag in Europe state that worn patches should be thrown away in such a manner that the release of active ingredient should be either delayed or prevented, because of their high content of ethinyl estradiol and norelgestromin. The instructions in this publication state that the worn patch should be put back into the primary package (four-edged sealed bag) and manually closed again or e.g. returned to the pharmacy, which throws out the patch separately as old medicine. For the German market recently the EVRA patch has been marketed with a separate disposal bag in the primary package-bag. The patch is glued after use to the outer surface of the sealed edge bag and a label or sticker is glued completely over the patch edges. Thus the patch is found in a structure like a pocket between the disposal label and the primary packaging. This structure however does not entirely prevent the release of active ingredient into the environment, but only delays it. Of course the migration of the effective ingredient through the aluminum layer of the packaging on the one side of the pocket and the polyester foil of the label on the other side is probably negligibly small. However a transverse diffusion of the effective ingredient through the label adhesive occurs to the outside open cut edges. Also the adhesive or glue only temporarily withstands the storage conditions in the house garbage, which eventually loosen the label, open the disposal pocket and thus release the active ingredient.

The special misuse risk of opioid-containing transdermal systems has led to technical disposal solutions as described and claimed in WO 02/085268 (Marceniak, et al, for Purdue Pharma L.P.). However the solution described in this reference is directed exclusively at the active ingredient, which is in danger of misuse, and to an inactivation or to making it unusable as much as possible immediately after use of the patch. The goal is to practically instantaneously eliminate the effectiveness or usability of the worn patch.

The suggested inactivation pathway includes especially the inactivation of the receptor binding site of the opioid or the binding of the opioid to the receptor for production of an insoluble complex (see p. 12, line 10 to 19; p. 14, lines 24 to 38 and p. 15, lines 5 to 13, of the WO reference cited above). In one concrete example for inactivation only the receptors, antibodies or antagonistic effective ingredients are named (see p. 15, lines 26 to 29). The inactivation mechanisms described in WO 02/085268 are very specific in nature with regard to opiod active ingredients or other effective ingredients with misuse potential. The types of conceivable inactivation mechanisms are explained in very general terms and include chemical physical, electrical or magnetic (see p. 15, lines 14 to 23). They are not described completely in the examples. Furthermore the inactivation relates to the use by humans. There are no suggestions regarding how to prevent the effective ingredient from acting on other living organisms or otherwise affecting the environment.

### SUMMARY OF THE INVENTION

It is an object of the present invention to develop a concept for disposal of worn or unworn but no longer required effective-ingredient-containing patches, in which the release of the residual amount of pharmaceutically active ingredient in the patch into the environment after throwing the patch in the house garbage is prevented by its chemical conversion or at least strongly reduced.

It is another object of the present invention to design the chemical transformation or conversion so that its product no longer has any relevant pharmacological or toxicological action.

It is a further object of the present invention to fashion the chemical transformation or conversion so that the products cannot be converted back by environmental influences, especially known microbiological activities, e.g. ester cleavage, to a pharmacologically active form again.

It is still another object of the present invention to provide means for chemical transformation or change so that for its part it produces no unacceptable pollution of the environment with pollutants or handling risk for laymen.

All the foregoing objects surprisingly could be attained when the formulations for making polymer films for cosmetic teeth whitening were applied to the described disposal problem for transdermal patches.

Nonspecific oxidation for removal of teeth coatings and discoloration are used in the teeth whitening area. The selection of chemically moderately reactive reactants, which are even suitable for use in the mouth and provide the required chemical stability for the chemically very reactive teeth whitening film during storage until it is used, is valued. In this connection especially U.S. Pat. No. 6,689,344 “Patches for Teeth Whitening”, issued to Chang, et al, for LG Household & Healthcare Ltd. Seoul, is cited. All the references named there are incorporated here by reference for the present invention. Information regarding suitable unspecific oxidizing agents and suitable polymer matrices, compatible auxiliary agents and stabilizing features, especially the formulations described in claims 1 to 25 of this patent, was taken from this reference.

The nonspecific chemical oxidizing agents are characterized as decomposition accelerators in the present invention, in the sense, that they speed up the oxidative chemical decomposition of the pharmaceutically active ingredients to pharmacologically and biologically inactive decomposition products. Decomposition products are considered to be inactive when they are at least a factor of 10, especially at least a factor of 100, weaker in the activity of the starting material. Besides direct self-oxidizing agents those substances which accelerate the oxidative decomposition processes of pharmaceutically active ingredients in the presences of air oxygen or direct oxidizing agents without self-oxidizing action can be used as decomposition accelerators. These substances include e.g. organic and inorganic metal salts of cooper, iron or manganese. Since oxidative decomposition rate frequently depends on the pH value, chemically basic or acid reacting materials can be used as decomposition accelerators.

Compounds containing chemically bound hydrogen peroxide or elemental iodine can be used as the directly oxidizing decomposition accelerators and at the same time environmentally acceptable oxidizing agents. These compounds include especially urea peroxide (adduct of urea and hydrogen peroxide), hydrogen peroxide-PVP adduct, PVP-iodine (Povidone-Iodine, Polividone-Iodine), alkali persulfate (e.g. potassium monopersulfate=OXONE®), alkali carbonate peroxide, calcium peroxide, zinc peroxide, alkali perborate or sodium pyrophosphate peroxide. Mildly oxidizing substances, such as alkali hydpochloride can be used, which e.g. are used to disinfect swimming pools. Moreover hydroperoxides and acid peroxides, such as peroxyacetic acid and benzoyl peroxide are usable. Peroxides sterically blocked by branched chemical groups are especially suitable peroxides and are especially stable. Hydroquinone or anthraquinone are additional mildly oxidizing additives.

In the case of peroxides stabilizing additive ingredients, especially alkyl sulfates like sodium dodecyl sulfate and the substances described in claim 16 of U.S. Pat. No. 6,689,344 or combinations of those substances, can also be used.

Especially bivalent and trivalent iron (Fe(II+), Fe(II+)), bivalent copper (Cu(II+)), bivalent, trivalent or quadrivalent manganese (Mn(II+), Mn(III+), Mn(IV+) or bivalent zinc (Zn(II+)) are considered for the oxidative decomposition promoting and at the same time environmentally acceptable metal ions. Their organic salts are suitable for embedding in lipophilic matrices because of the contrasting high solubility of their inorganic salts.

Particularly dispersed insoluble additives or soluble alkaline-reacting additives are suitable for lipophilic polymer films to accelerate decomposition by shifting the pH into the alkaline range. Largely insoluble dispersible additive ingredients for lipophilic polymer films are selected from the group consisting of alkali silicates, such as sodium trisilicate (water glass) or sodium metasilicate, and tribasic sodium phosphate, sodium carbonate and potassium carbonate. Soluble lipophilic additives are preferably selected from the group consisting of Eudragit E100 (Polyacrylate with basic reacting side chains), diethanolamine, triethanolamine, TRIS, a minomethylpropanol and its N-alkyl derivatives, ethylenediamine, arginine and lysine.

Especially soluble acid-reacting additives are suitable for lipophilic polymer films to accelerate decomposition by shifting the pH to the acid range. Soluble acid reacting additives are preferably selected from the group consisting of monovalent to trivalent organic acids with a pKₐ value of less than 4, for example lactic acid, citric acid, tartaric acid, succinic acid, fumaric acid and malic acid.

While the moist tooth surface solvates a non-self-adhering polymer film based on a water-soluble polymer on contact and because of that the polymer film adheres to it, the transdermal patch forms no adherent bonds with a water-soluble polymer film because of its strong lipophilic character. Furthermore a typical lipophilic ingredient is transported only slowly from the transdermal patch into a hydrophilic aqueous layer, since the patches are optimized for release of the active ingredient into the lipophilic outer skin layer.

The concept of the hydrophilic polymers for teeth whitening, e.g. from U.S. Pat. No. 6,689,344, was converted to lipophilic polymers films for chemical disposal of transdermal active ingredient patches. The adhesive properties of the patch due to the adhesive residue are too greatly reduced after removal from the skin, in order to provide a completely adherent bond with a flat substrate. For this reason the polymer film according to the invention is preferably formulated with a lipophilc adhesive, so that this polymer film can form a completely adherent bond to the patch.

Adhesives, which have no or only a few functional groups and thus are nonreactive or have only negligible chemical reactivity, are selected because of the content of chemically reactive substances. These adhesives include, e.g., those based on hydrocarbon polymers, such as polyisobutylene, polybutene, polyisoprene or styrene block copolymers, such as SIS and SBS. Silicone polymers are similarly especially suitable because they are largely chemically inert. However they are generally too expensive for the present purposes. Acrylates, especially high molecular weight acrylates, which can be used without chemical cross-linking systems by means of functional groups, are suitable as the adhesive. For example, these latter acrylate adhesives include Duro-Tak 87-9301 and/or Duro-Tak 387-2051 and Duro-Tak 387-2353, which only have a predominantly oxidation-resistant carboxyl group. In contrast, acrylate adhesives with alcoholic groups in their side chains, such as Duro-Tak 387-2287, are less suitable. Acrylate adhesives with alcoholic groups, such as Duro-Tak 387-2287 and Duro-Tak 387-4287, generally can be especially suitable when a decomposition accelerator is worked into the film, which does not directly cause oxidation, such as metal ions or additives that shift pH.

Furthermore polymer films, which have high solubility for the effective ingredient in contrast to the effective-ingredient containing matrix of the transdermal patch, are preferred as the decomposer film. In this way the effective ingredient is transferred especially rapidly and completely into the decomposer film. A decomposer film based on acrylates is especially preferred for use together with a steroid hormone-containing patch on the basis of a hydrocarbon adhesive or a silicone adhesive, since the steroid hormone, e.g. ethinyl estradiol, has a higher solubility in acrylate adhesives than in polyisobutylene or silicone adhesives.

Highly dispersed filling materials, e.g. silicon dioxide (aerosol types from Degussa), talcum powder, zinc oxide or titanium dioxide, preferably as nanoparticles, such as those used e.g. as light-protective agents in sun blocking compositions, can be used to increase the interior surface area and thus further accelerate the decomposition reaction. Furthermore activated charcoal, which adsorptively binds the active ingredient besides increasing surface area, is suitable as a filling material.

### BRIEF DESCRIPTION OF THE DRAWING

The objects, features and advantages of the invention will now be illustrated in more detail with the aid of the following description of the preferred embodiments, with reference to the accompanying figures in which:

FIGS. 1a and 1b are respective cross-sectional views of a first embodiment of a marketed decomposer film product according to the invention with supporting liner and of a worn (used) or unused effective ingredient-containing patch enclosed between the decomposer film and a carrier sheet;

FIGS. 2a and 2b are respective cross-sectional views of a second embodiment of a marketed decomposer film product according to the invention with supporting liner and of a worn (used) or unused effective ingredient-containing patch enclosed between the decomposer film and the supporting liner;

FIGS. 3a and 3b are respective cross-sectional views of a third embodiment of a marketed decomposer film product according to the invention with supporting liner and of a worn (used) or unused effective ingredient-containing patch enclosed by the decomposer film without a carrier sheet; and

FIGS. 4a and 4b are respective cross-sectional views of a fourth embodiment of a marketed decomposer film product according to the invention with supporting liner and decomposer cover with barrier layer and of a worn (used) or unused effective ingredient-containing patch enclosed between the decomposer film and a carrier sheet.

### DETAILED DESCRIPTION OF THE INVENTION

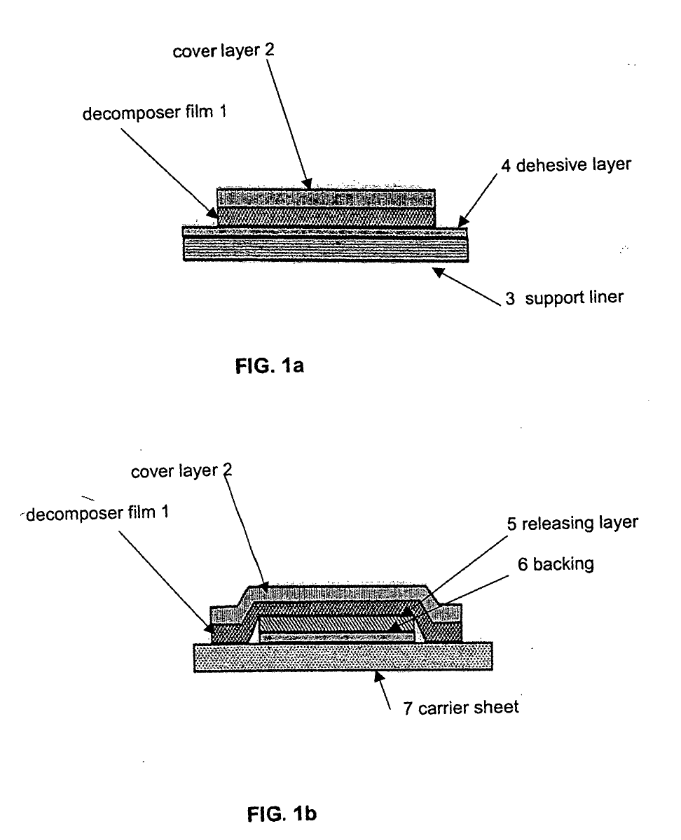

The decomposer film product shown in FIG. 1a comprises a self-adherent decomposition-accelerating polymer film 1 arranged between a cover layer 2 acting to cover the film from the outside and a support liner or sheet 3, which acts as a flat support. The support liner or sheet 3 is provided with a dehesive protective layer 4 between it and the decomposer film 1, which permits removal of the decomposer film 1 and its cover layer 2 from the support sheet 3 prior to use.

FIG. 1b shows the decomposer film 1 of FIG. 1a in use. The decomposition-accelerating polymer film 1 is shown in full contact with the effective-ingredient-releasing layer 5 of the effective-ingredient-containing patch. The decomposer film 1 has a greater surface area than that of the releasing layer 5 delivering the effective ingredient, so that the outer effective-ingredient-releasing layer edges are covered on all sides by the decomposer film 1 as shown in FIG. 1b. In this embodiment the effective-ingredient-containing patch comprises a single matrix layer 5 and a backing 6 as an additional layer besides the effective-ingredient-containing matrix layer. Understandably the effective-ingredient-containing patch can also have multiple layers or a reservoir structure according to the state of the art, as long as the surface delivering the effective ingredient is in direct contact with the decomposition accelerating film 1. The patch is fixed on a carrier sheet 7 by means of the adhesive edge regions of the decomposer film 1 that extend beyond the patch effective-ingredient or drug-releasing layer 5. The carrier sheet 7 is a flat support, which is part of the primary or secondary packaging for the patch or an insert in the package in which the patch is marketed. For example, it can be supported on the interior or outer surface of the four edged sealed bag, a deep-drawn package (primary package), a surrounding packet (secondary package), a booklet, a patient information or special disposal card. However it can also be the dehesively equipped support liner 3.

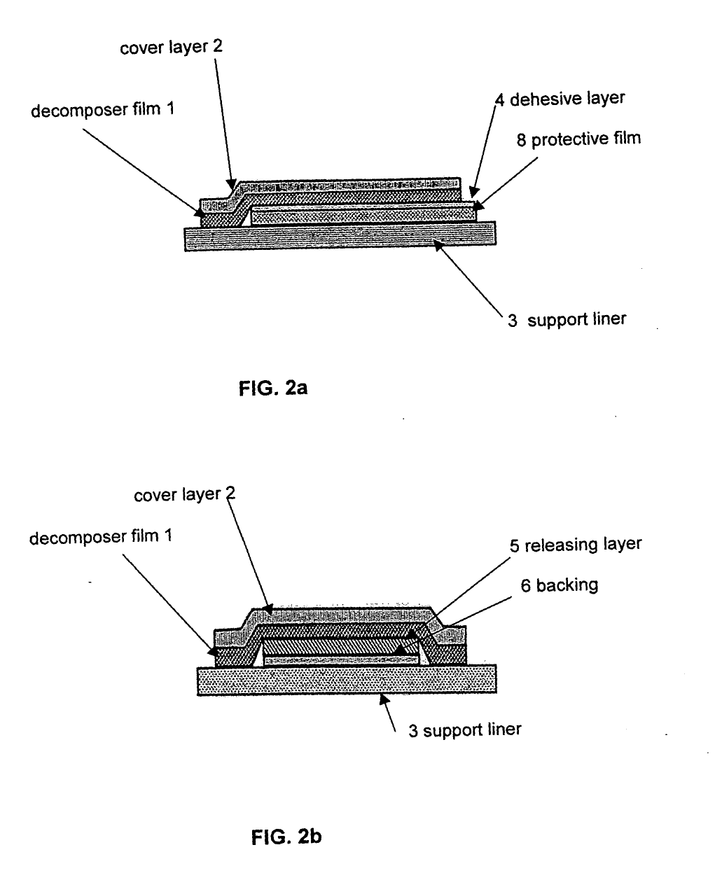

Another embodiment of the decomposer film product shown in FIG. 2a comprises a self-adherent decomposition-accelerating polymer film 1 arranged between a cover layer 2 acting to cover the decomposer film from the outside and a support liner 3, which acts as a flat support. The support liner 3 is a sheet, which is not provided with a dehesive protective layer as in the case of the embodiment shown in FIG. 1a. Furthermore adhesive bonding between the decomposer layer 1 and the support liner 3 is largely avoided by an intervening protective film 8, which has a dehesive layer 4 on its surface facing the decomposer film 1. An adhesive bond between the decomposer film 1 and the support liner 3 is formed on only one edge region.

FIG. 2b shows the decomposer film 1 of FIG. 2a in use. The decomposition-accelerating polymer film 1 is shown in full contact with the effective-ingredient-releasing layer 5 of the effective ingredient patch. The decomposer film 1 has a greater surface area than that of the patch layer 5 delivering the effective ingredient, so that the outer effective-ingredient-releasing layer edges are covered on all sides by the decomposer film 1. In this embodiment the effective-ingredient containing patch is a single matrix layer 5, which has a backing 6 as an additional layer besides the effective-ingredient-containing matrix layer 5. Understandably the effective-ingredient-containing patch can also have multiple layers or a reservoir structure according to the state of the art, as long as the surface delivering the effective ingredient is in direct contact with the decomposition accelerating film 1. The patch is fixed on support liner 3 by means of the adhesive edge regions of the decomposer film 1 that extend beyond the patch effective-ingredient or drug-releasing layer 5. However in the case of this embodiment in contrast to that shown in FIG. 1b a permanent non-releasable adhesive bond between the decomposer film 1 and the support liner 3 results because of the absence of the dehesive layer. This structure provides additional protective action that helps to prevent effective ingredient release to the environment, since the adhesive bond around the effective-ingredient-containing patch is permanent.

The carrier sheet 7, on the other hand, is a flat support, which is a part of the primary or secondary packaging or an insert in the packaging of the patch. For example, it can be supported on the interior or outer surface of a four edged sealed bag, a deep-drawn package (primary package), a surrounding packet (secondary package), a booklet, a patient information or special disposal card.

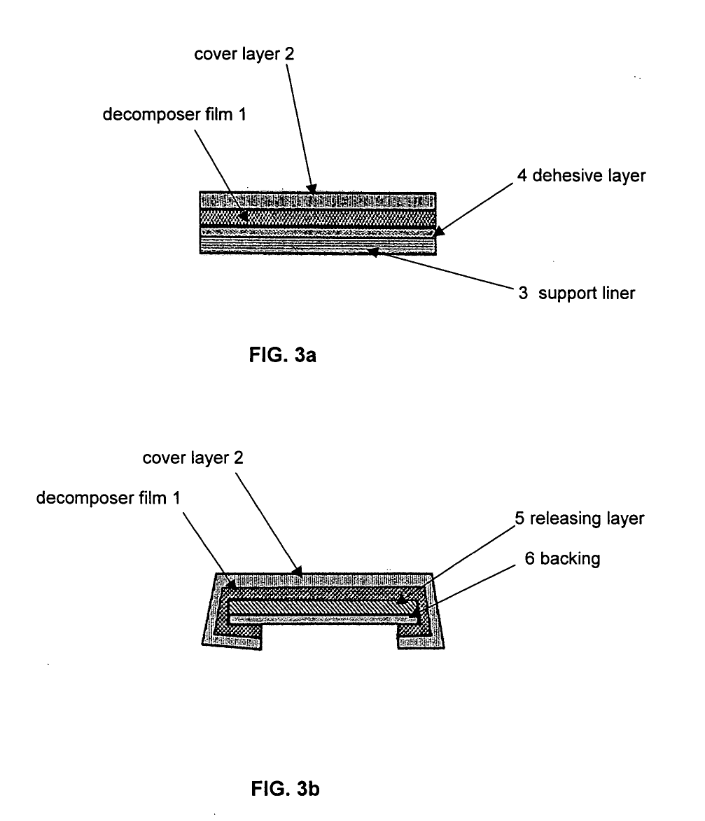

A further embodiment of the decomposer film product shown in FIG. 3a comprises a self-adherent decomposition-accelerating polymer film 1 arranged between a cover layer 2 acting to cover the decomposer film from the outside and a support liner 3, which acts as a flat support. The support liner 3 is provided with a dehesive protective layer 4, which allows it to be removed from the decomposer film and the decomposer cover prior to use.

FIG. 3b shows the decomposer film 1 of FIG. 3a in use. The effective ingredient-containing patch in this embodiment is a single layer matrix system comprising a matrix layer 5 and a backing 6. The decomposition-accelerating polymer film 1 is shown in full contact with the effective-ingredient-releasing layer 5 of the effective-ingredient-containing patch in FIG. 3b. The decomposer film 1 has a greater surface area than that of the effective-ingredient-containing patch, so that the edge regions of the decomposer film 1 can extend around and adhere to the edges of the effective-ingredient containing patch, as shown in FIG. 3b. In this embodiment the release of effective ingredient from the patch edges is prevented without the use of an underlying carrier sheet or support liner as a substrate, which is required in FIGS. 1a to 2b.

The decomposer cover layer is preferably oxygen permeable, in order to promote the admission of air oxygen to the combination of the decomposer film and the effective-ingredient-containing patch and thus oxidation decomposition processes. In this connection preferred embodiments of the cover sheet are made from respiration active textile fabrics or fleece materials and further oxygen permeable foils, such as polyurethanes or cellulose derivatives.

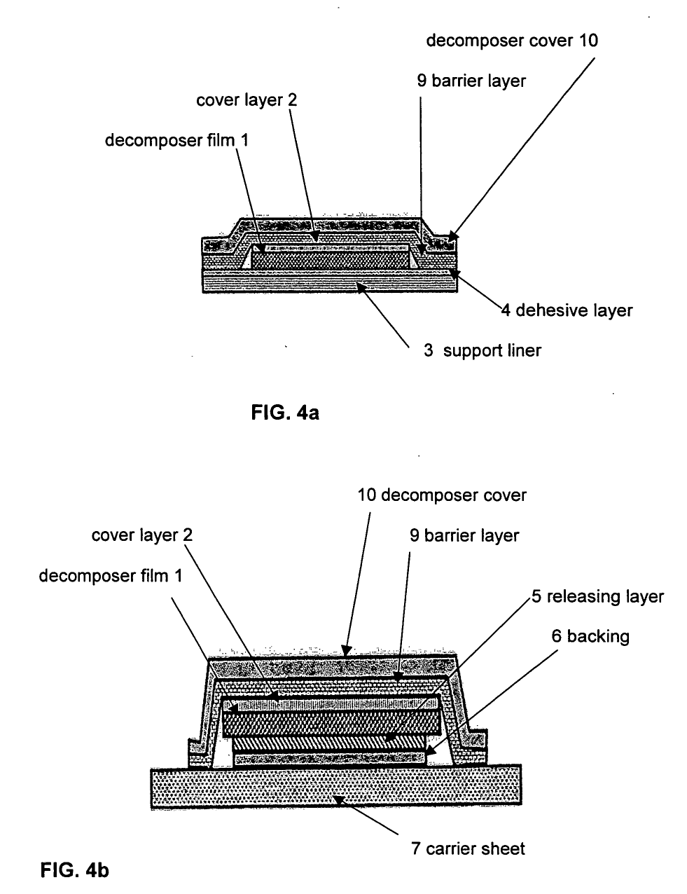

A further embodiment of the decomposer film product shown in FIG. 4a is the same as the embodiment shown in FIG. 1a, except that the decomposer film product has additional protective layers over it. The decomposer film 1 and cover layer 2 in this embodiment are protected from the outside by a decomposer cover 10 and a barrier layer 9. This embodiment is particularly useful in the case of decomposition accelerating agents comprising peroxides, since the barrier layer 9 prevents admission of air and moisture prior to use and thus extends the storage life of the decomposer film. The barrier layer can be barrier foil, such as PET or PVC, to reduce the admission of air and moisture.

According to the type of the decomposition accelerating additive, especially peroxides or iodine-containing materials, the embodiment shown in FIGS. 4a and 4b may be necessary because of reduced chemical stabilities to provide a primary package for the decomposition film in its state prior to use according to FIGS. 1a to 3b, which protects against admission of light, air and moisture or against the escape of oxygen or iodine.

FIG. 4b shows the decomposer film 1 of FIG. 4a in use. The operation of this embodiment is basically the same as that of FIG. 1b, except that the decomposer film 1 is held in pace on the effective-ingredient-containing patch or transdermal patch by adhesive edge regions of the barrier layer 9. For this purpose in this embodiment the decomposer cover 10 and barrier layer 9 have an area that is greater than that of the decomposer film 1 and cover layer 2.

The following examples serve to illustrate the claimed invention but their details should not be considered as limiting the appended claims.

### EXAMPLARY FORMULATIONS FOR THE DECOMPOSER FILM

The examples describe preparations of coating solutions and making polymers films of the invention containing one or more decomposition accelerators (decomposers) using the coating solutions.

The manufacture of polymers films occurs by coating of a suitable release liner, e.g. siliconized Hostaphan 100 μm polyester foils or coating of paper coated with polyethylene. The drying occurs for 10 minutes at 80° C. in a suitable air circulating drying chamber after ventilating at room temperature. However examples 6, 7 and 8 (containing peroxide and iodide) below utilize a somewhat different drying procedure because of hydrogen peroxide and/or iodine cleavage. In these latter examples, after ventilation, drying occurred for 20 minutes at only 50° C. The desired flat weight of the dried film is 50 g/m²; however 10 to 100 g/m² can be used. The dried films are covered with a thin polyester foil, e.g. Hostaphan RN 12. Alternatively for this purpose air and oxygen permeable materials from the group consisting of woven and non-woven fleece materials or oxygen permeable polymer foils, such as polyurethane foils or foils from cellulose derivatives, are used.

### Example 1

### Fe(III) Citrate in Lipophilic Acrylate Film with about 0.5% (g/g) Fe(III) Citrate

| | Parts by Weight in dry film |
|---|---|
| Fe(III) citrate - preliminary solution | 0.5 |
| Durotak 387-2052 | 99.5 |

A 10% (g/g) mixture of the decomposer Fe(III) citrate in water is prepared and the citrate is dissolved by heating to a slow boil to form the preliminary solution. The required amount of this preliminary solution is directly added dropwise in Durotak and stirred in uniformly. A turbid, orange-colored solution (further processed as fresh as possible and protected from sunlight) is produced.

### Example 2

### Fe(III) Citrate in Water-Soluble Methacrylate Film with about 0.5% (g/g) Fe(III) Citrate

| | Parts by Weight in dry film |
|---|---|
| Fe(III) citrate - preliminary solution | 0.5 |
| Ethanol | 16 |
| Adhesive Solution Roehm E35H | 99.5 |

The preliminary solution of the decomposer Fe(III) citrate is prepared as above. The required amount of preliminary solution is diluted next with 1+9 (g/g) parts ethanol (turbidity therein). The E35H is weighed into this mixture and stirred uniformly until yellowish particles dissolve again completely (which takes about a few hours). A canary yellow clear solution arises—further processed as much the same as possible and not stored in sunlight.

### Example 3

### Fe(III)-Citrate-Lipophilic Alkali Acrylate Film with about 1.0% (g/g) Fe(III) Citrate—Completely Dissolved

| | Parts by Weight in dry film |
|---|---|
| Fe(III) citrate - preliminary solution | 1.0 |
| Aminomethylpropanol | 5.7 |
| Durotak 387-2051 | 84.3 |

A 10% (g/g) solution of the decomposer Fe(III) citrate acting as a preliminary solution is placed in water and dissolved by heating to boiling. Aminomethylpropanol was weighed into the Durotak adhesive and stirred. The viscosity increase was compensated by adding as much methanol as needed (about 5 ml methanol to 10 ml of Durotak). Finally the required amount of the iron salt solution was added drop-wise and stirred. A rapidly clearing orange emulsion (further processed as fresh as possible and protected from sunlight) is produced.

### Example 4

### Mn(III) Acetate or Cu(III) Acetate Decomposer Dissolved in an Adhesive

| | Parts by Weight in dry film |
|---|---|
| Decomposer | 1 |
| Durotak 387-2353 | 99 |

Decomposer was prepared as a 10% solution in ethanol and the required amount of Durotak directly stirred in.

### Example 5

### Mn(IV) Oxide (Manganese Dioxide) Dispersed in an Adhesive

| | Parts by Weight in dry film |
|---|---|
| Manganese dioxide | 1.0 |
| Durotak 387-2353 | 99 |

Manganese dioxide is slurried with some ethyl acetate (about 1 MnO₂+2 parts EtOAc) and adhesive and stirred uniformly.

### Example 6

### Iodine-PVP in an Adhesive

| | Parts by Weight in dry film |
|---|---|
| Iodine-PVP | 5 |
| Durotak 387-2051 | 95 |

A 30% PVP Iodine solution is methanol is prepared. The required amount of this solution is stirred into the Durotak. The possibly critical viscosity increase can be compensated as needed by dilution with ethyl acetate.

### Example 7

### Urea Peroxide in an Adhesive

| | Parts by Weight in liquid |
|---|---|
| | Adhesive Mass |
| Preliminary solution | 40 |
| Durotak 287-2287 | 80* |
| Ethyl acetate | 10 |
| Heptane | 5 |
| | Parts by Weight in Liquid |
| | Preliminary Solution |
| Collidone 30 | 30 |
| Methanol | 40 |
| Urea Peroxide | 10 |

*was made with Durotak 2287 of about 40% solids content.

The collidone is dissolved in methanol and 10 parts of urea peroxide are dissolved in this solution with stirring in a loosely closed vessel to make the preliminary solution. Then the required amount of the preliminary solution is weighed into the Durotak 2287 present, and the ethyl acetate and heptane are added and the resulting mixture is uniformly stirred.

### Example 8

### Oxone (Potassium Persulfate)

| | Parts by Weight in liquid |
|---|---|
| | Adhesive Mass |
| Preliminary solution | 15 (corresponds to about |
| | 5% solid oxone) |
| Durotak 387-2353 | 85* |
| Ethyl acetate q.s. | (for dilution as needed) |
| | Parts by Weight in |
| | Liquid Preliminary Solution |
| Oxone | 15 |
| Sodium lauryl sulfate | 10 |
| Glycerol | 30 |
| Water | 45 |

*relates to an about 37% solids content.

The oxone is dissolved in water, sodium lauryl sulfate and glycerol are added and the resulting mixture is uniformly stirred (moderate stirring, since otherwise large amounts of foam are generated). The required amount of the preliminary solution is weighed into the Durotak and the resulting mixture is stirred to form a uniform emulsion.

### Example 9

### Combination System Manganese Dioxide+Fe III

| | Parts by Weight in dry film |
|---|---|
| Iron (III) citrate | 0.5 |
| Manganese dioxide | 0.5 |
| Durotak 387-2051 | 99 |

The manufacture occurs analogously to example 1, however manganese dioxide is added to the finished product of example 1 by weighing in the manganese oxide powder and stirring in the adhesive until a uniform orange-gray suspension is produced.

### Example 10

### Alkaline Film 1

| | Parts by Weight in dry film |
|---|---|
| Sodium silicate | 10 |
| Durotak 387-2287 | 90 |

Sodium trisilicate (water glass) is ground in a hammer bar mill and screened by a screen with a mesh width in a range from 25 to 50 μm. The obtained powder is slurried with ethyl acetate (about 1+1 part) and added to the Durotak. The mixture is stirred to form a homogeneous suspension.

### Example 11

### Alkaline Film 2

| | Parts by Weight in Dry Film |
|---|---|
| Trisodium phosphate (12 H₂O) | 5 |
| Glycerol | 10 |
| Durotak 387-2051 | 85 |
| | Parts by Weight in Liquid |
| | Preliminary Solution |
| Trisodium phosphate (12 H₂O) | 5 |
| Water | 10 |
| Glycerol | 10 |

Sodium phosphate is dissolved in water and glycerol added. The necessary amount of the pre-solution is stirred into Durotak until a uniform emulsion arises.

### Example 12

### Combination of Decomposition Accelerators in a Hydrocarbon Adhesive

| | Parts by Weight in dry film |
|---|---|
| Copper (II) acetate 1 | 1 |
| MnO₂ (Manganese dioxide) | 1 |
| MA24A | 98 |

A 5% by weight solution of copper (II) acetate in methanol is prepared and the required amount of this solution is stirred directly into the adhesive solution MA24A of Adhesives Research Inc.—manganese dioxide is scattered in and uniformly stirred.

### Example 13

### Combination of Decomposition Accelerators with Nanoparticles in an Adhesive Film

| | Parts by Weight in dry film |
|---|---|
| Mn (III) acetate | 1 |
| TiO₂ | 1 |
| Durotak 387-2287 | 98 |

Titanium dioxide nanoparticles are slurried with ethyl acetate (about 1 part nanoparticles+2 parts ethyl acetate and weighed into the Durotak solution. A 5% solution of Mn (III) acetate in ethanol is prepared and the required amount of this solution is directly stirred into the mixture of Durotak and nanoparticles. The resulting mixture is uniformly stirred.

### Example 14

### Combination of Decomposition Accelerators and Acid pH Regulator

| | Parts by Weight in dry film |
|---|---|
| Mn (III) acetate | 1 |
| Lactic Acid | 1 |
| Durotak 387-2353 | 98 |

A 5% solution of Mn(III) acetate in ethanol is prepared and the required amount of this solution is stirred into a mixture of Durotak and lactic acid.

### Example 15

### Combination of Decomposition Accelerates and Basic pH Regulator

| | Parts by Weight in dry film |
|---|---|
| MnO₂ (Manganese dioxide) | 1 |
| Triethanolamine | 5 |
| Durotak Elite 87-9301 | 94 |

The manganese dioxide was slurried with ethyl acetate (about 1 part manganese dioxide and 1 part of EtOAc) and added to the adhesive solution, triethanol amine was weighed in the resulting mixture was uniformly stirred.

At all places in the appended claims at which an effective ingredient is mentioned, this relates not only to a transdermal patch with a single effective ingredient but also to a patch with two effective ingredients. In the case of a patch with a combination of two effective ingredients the operation of the decomposition accelerators can be directed to only one or both effective ingredients.

While the invention has been illustrated and described as embodied in a decomposer film for transdermal patches, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A polymer film comprising at least one decomposer layer, wherein said at least one decomposer layer contains at least one decomposition accelerator, said decomposition accelerator being effective to decompose at least one pharmaceutical effective ingredient contained in a worn or unused transderamal patch, when an effective ingredient releasing layer of said patch adheres to the polymer film so that the pharmaceutical effective ingredient comes into contact with the decomposition accelerator by diffusion.

2. The polymer film as defined in claim 1, wherein at least 50 percent by weight of the at least one pharmaceutical effective ingredient in the transdermal patch has decomposed chemically one month after adherence of the effective ingredient releasing layer to the polymer film and at least 90 percent by weight of the at least one pharmaceutical effective ingredient has decomposed chemically six months after adherence of the effective ingredient releasing layer to the polymer film.

3. The polymer film as defined in claim 1, in which the at least one pharmaceutical effective ingredient has a solubility in said at least one decomposer layer that is greater than a solubility of the at least one pharmaceutical effective ingredient in the effective ingredient releasing layer of the transdermal patch.

4. The polymer film as defined in claim 3, wherein said solubility in said decomposer layer is at least twice as great as said solubility in said releasing layer of the transdermal patch.

5. The polymer film as defined in claim 1, wherein said at least one decomposition accelerator comprises a chemically oxidizing substance.

6. The polymer film as defined in claim 5, wherein said chemically oxidizing substrate is selected from the group consisting of iron (III) citrate, manganese dioxide, manganese (III) acetate, copper (II) acetate, urea peroxide, carbamide peroxide, potassium persulfate, sodium persulfate, potassium hypochlorite, sodium hypochlorite, sodium percarbonate, sodium polyphosphate peroxide, zinc peroxide and peroxy acetic acid.

7. The polymer film as defined in claim 5, wherein said chemically oxidizing substrate is selected from the group consisting of urea peroxide, manganese (III) acetate and iron (III) citrate.

8. The polymer film as defined in claim 5, wherein said at least one decomposition accelerator comprises an alkaline reacting substance.

9. The polymer film as defined in claim 8, wherein said alkaline reacting substance is potassium hydroxide, sodium hydroxide, triethanolamine, diethanolamine, 2-amino-2-methyl-1-propanol, hydrotalcide, arginine, lysine, Eudragit E100, sodium trisilicate, sodium metasilicate, disodium hydrogen phosphate, trisodium phosphate or sodium polyphosphate ((NaPO₃)₆.

10. The polymer film as defined in claim 8, wherein said alkaline reacting substance is selected from the group consisting of 2-amino-2-methyl-1-propanol, sodium trisilicate and sodium metasilicate.

11. The polymer film as defined in claim 1, wherein said at least one decomposition accelerator comprises at least one catalytically acting metal ion.

12. The polymer film as defined in claim 11, wherein said at least one catalytically acting metal ion is selected from the group consisting of iron (III), copper (II) and Manganese (III) and said at least one catalytically acting metal ion is present in the polymer film in the form of at least one organic or inorganic salt.

13. The polymer film as defined in claim 12, wherein said at least one organic or inorganic salt is present in dissolved form.

14. The polymer film as defined in claim 1, wherein said at least one decomposition accelerator comprises at least one chemically acid reacting substance.

15. The polymer film as defined in claim 14, wherein said at least one chemically acid reacting substance is at least one organic acid with a pKₐ value under 4.0.

16. The polymer film as defined in claim 15, wherein said at least one organic acid is selected from the group consisting of lactic acid, ethylenediamine tetracetic acid, tartaric acid, succinic acid and citric acid.

17. The polymer film as defined in claim 1, wherein said at least one decomposition accelerator comprises a plurality of decomposition accelerating agents.

18. The polymer film as defined in claim 1, having adhesive properties and based on a water-insoluble adhesive.

19. The polymer film as defined in claim 18, wherein said water-insoluble adhesive is a hydrocarbon material or an acrylate adhesive.

20. The polymer film as defined in claim 1, wherein said at least one pharmaceutical effective ingredient is at least one steroid hormone selected from the group consisting of gestagens, estrogens and androgens.

21. The polymer film as defined in claim 20, wherein said at least one steroid hormone is ethinyl estradiol, levonorgestrel or gestoden.

22. The polymer film as defined in claim 1, further comprising a flat support liner and a releasable protective film for covering the polymer prior to use.

23. The polymer film as defined in claim 22, wherein said releasable protective film is on a side opposite to that of said flat support liner.

24. The polymer film as defined in claim 22, wherein said flat support linear is part of a primary or secondary package of the transdermal patch.

25. The polymer film as defined in claim 19, wherein said hydrocarbon material is selected from the group consisting of polyisobutylene, polyisoprene and polybutene.

26. The polymer film as defined in claim 1, having no adhesive properties and based on a water-soluble adhesive.

27. The polymer film as defined in claim 26, wherein said water-soluble adhesive is selected from the group consisting of polyvinyl alcohol, polyvinyl pyrrolidone, hydroxypropyl cellulose, hydroxypropylmethyl cellulose, carboxymethyl cellulose and polyacrylic acids.

28. The polymer film as defined in claim 1, wherein the at least one decomposition accelerator is completely dissolved in said at least one decomposer layer.

29. The polymer film as defined in claim 1, comprising a highly dispersed filling material for increasing interior surface area.

30. The polymer film as defined in claim 29, containing from 0.1 to 5.0 percent by weight of said highly dispersed filling material and said highly dispersed filling material is selected from the group consisting of silicon dioxide, titanium dioxide, zinc oxide, talcum powder and activated charcoal.
